(19) United States
(12) Patent Application Publication
(10) Pub. No.: US 2025/0000793 A1
(43) Pub. Date: Jan. 2, 2025

(54) USE OF NANOPARTICLES FOR TREATING RESPIRATORY INFECTIONS

(71) Applicant: EVOQ NANO, INC., Salt Lake City, UT (US)

(72) Inventor: William H. Niedermeyer, West Jordan, UT (US)

(21) Appl. No.: 18/884,646

(22) Filed: Sep. 13, 2024

Related U.S. Application Data
(63) Continuation of application No. 16/926,199, filed on Jul. 10, 2020, now Pat. No. 12,115,250.
(60) Provisional application No. 62/873,516, filed on Jul. 12, 2019.

(51) Int. Cl. A61K 9/00; A61K 9/08; A61K 9/51; A61K 33/38; A61M 15/00

(52) U.S. Cl. CPC A61K 9/0078; A61K 9/5115; A61K 33/38; A61M 15/0085; A61K 9/08

(57) ABSTRACT

This disclosure relates to silver nanoparticle compositions and methods for treating respiratory infections, such as one or more of a bacterial, fungal, or viral infection. A treatment composition comprising nonionic, ground state silver nanoparticles and a carrier is administered to a patient's lungs via inhalation. The silver nanoparticles have properties that enable effective transport through biofilm and mucus layers to epithelia and surrounding tissues, killing or deactivating microbes at the targeted respiratory tissue and throughout the overlying mucus layer without the release of silver ions and without damaging respiratory tissue.

### CROSS-REFERENCE TO RELATED APPLICATIONS

This application is a continuation of United States Patent Application No. 16/926, 199, filed Jul. 10, 2020, which claims the benefit of U.S. Provisional Application No. 62/873,516, filed Jul. 12, 2019, each of which is incorporated herein by this reference in its entirety.

### BACKGROUND

Cystic fibrosis is an inherited condition where the cystic fibrosis transmembrane conductance regulator (CFTR) proteins become dysfunctional due to mutations in the CFTR gene. Without proper CFTR protein function, mucus in various organs becomes thick, sticky, and difficult to properly clear. In the respiratory system, and particularly in the lungs, the thick mucus tends to gather and trap bacteria and other microbes, leading to frequent and/or chronic respiratory infections.

Cystic fibrosis patients are prone to lung infections from multiple infections microbial agents, including known drug resistant bacteria such as Burkholderia cepacia and Pseudomonas aeruginosa species. These infections are challenging to treat in cystic fibrosis patients due to the viscous mucus that accumulates in the central airways and because bacteria form biofilms for self-protection. Once infected, cystic fibrosis patients are at risk of experiencing a rapid decline in lung function that can lead to severe lung disease and possibly death.

Conventional antibiotics cannot easily penetrate the thick mucus and biofilm layers, so administration via inhalation often fails to reach the underlying respiratory tissue and/or bacteria in the biofilm layers. On the other hand, systemic administration of antibiotics may eventually reach infected epithelia, but because more bacteria reside within the thick overlying mucus, reinfection readily occurs.

Accordingly, there is an ongoing need for compositions and methods for treating respiratory conditions associated with cystic fibrosis, and in particular for compositions and methods capable of effectively treating drug resistant bacterial infections commonly affecting cystic fibrosis patients.

### BRIEF SUMMARY

This disclosure is directed to compositions and methods for treating respiratory infections, such as respiratory infections associated with cystic fibrosis. In one embodiment, a treatment composition comprises a plurality of nonionic, ground state, spherical nanoparticles with no external edges or bond angles mixed in or mixable within a carrier formulated for administration to a patient via inhalation.

The treatment compositions described herein are able to effectively penetrate thick, viscous mucus layers to reach targeted microbes within the mucus and to reach underlying respiratory tissue. This beneficially allows the treatment composition to reach and treat underlying infected respiratory tissue. In addition, it allows the treatment composition to reach bacteria within the mucus and associated biofilm layers which can shield bacteria from conventional antibiotics. Notwithstanding the effective penetrative abilities of the nanoparticles of the treatment compositions described herein, they are also capable of being effectively cleared from the patient through normal clearance routes and thereby avoid building up within the treated respiratory tissue or other tissues or organs of the body.

In one embodiment, a method of treating a respiratory infection comprises administering the nanoparticle treatment composition to a patient via inhalation, and the treatment composition treating the respiratory infection. The infection may be, for example, caused by one or more antibiotic resistant bacteria. The treatment composition is beneficially able to kill or deactivate bacteria associated with the infection without harming respiratory epithelia and other nearby tissues.

The metal nanoparticles kill bacteria without significant release of silver (Ag+) or other metal ions. Because the metal nanoparticles do not release significant quantities of silver or other metal ions, they are essentially non-toxic to humans and other animals (i.e., whatever amount or concentration of ions, if any, that are released from the metal nanoparticles is/are below a threshold toxicity level at which they become toxic to humans, other mammals, birds, reptiles, fish, and amphibians).

In some embodiments, the nanoparticles are spherical and have a mean diameter of about 1 nm to about 40 nm, or about 2 nm to about 20 nm, or about 3 nm to about 15 nm, or about 4 nm to about 12 nm, or about 6 nm to about 10 nm, or a size range with endpoints defined by any two of the foregoing values. Nanoparticles within these size ranges, in particular nanoparticles having a mean diameter of about 8 nm, have been found to effectively penetrate mucus while still being capable of effective clearance from the patient's body (e.g., via the lymphatic system and kidneys).

The nanoparticles may be provided in an amount such that when mixed with the carrier, the nanoparticles have a concentration of about 10 ppb to about 100 ppm, or about 50 ppb to about 50 ppm, or about 200 ppb to about 20 ppm, or about 500 ppb to about 10 ppm, or about 1 ppm, or a concentration within a range defined by any two of the foregoing values.

Within these concentration ranges, the nanoparticles have been found to be effective in killing or deactivating targeted microbes. Beneficially, because the nanoparticles are effective even at relatively low concentrations, more dilute doses may be administered (and/or less nanoparticles may be dosed overall), which lowers the clearance burden on the body and reduces the risk of unwanted side-effects such as harm to the patient's own cells/tissues or systemic harm to other beneficial microbiota of the patient.

The treatment composition may be administered using any suitable inhalation route, including through the use of a metered-dose inhaler, a nebulizer, and/or a dry powder dispersion device. These types of devices typically include a mouthpiece or facemask enabling transfer of nebulized/atomized medicament to the patient. A nebulizer may be an ultrasonic nebulizer, a jet nebulizer, a vibrating mesh nebulizer, or a soft mist inhaler, for example.

The treatment compositions have shown versatile efficacy in treating a wide variety of bacteria, including several problematic bacterial strains that have resistance to one or more conventional antibiotics.

### BRIEF DESCRIPTION OF THE DRAWINGS

FIGS. 1A-1D show TEM images of various non-spherical nanoparticles (i.e., that have surface edges and external bond angles) made according to conventional chemical synthesis or electrical discharge methods;

FIGS. 2A-2C show TEM images of exemplary nonionic spherical-shaped metal nanoparticles (i.e., that have no surface edges or external bond angles), the nanoparticles showing substantially uniform size and narrow particle size distribution, smooth surface morphology, and solid metal cores without the use of coating agents;

FIGS. 3A-3C show transmission electron microscope (TEM) images of nonionic coral-shaped nanoparticles;

FIGS. 4A-4C schematically illustrated a proposed mechanism of action by which the nanoparticles can kill or deactivate bacteria; and

FIG. 5 illustrates the results of conductivity testing comparing various nanoparticle solutions and showing that spherical, metal nanoparticles according to the disclosed embodiments are nonionic.

### DETAILED DESCRIPTION

### Introduction

The present disclosure is directed to compositions and methods for treating respiratory infections, and in particular for treating respiratory infections associated with cystic fibrosis. In one embodiment, a treatment composition comprises a plurality of nonionic, ground state, spherical nanoparticles with no external edges or bond angles mixed in or mixable within a carrier formulated for administration to a patient via inhalation.

Although the present disclosure will often describe treatment of bacteria specifically, it will be understood that the same compositions and methods may additionally or alternatively be utilized to treat respiratory conditions that involve a viral and/or fungal infection, and the nanoparticle compositions described herein have shown efficacy against viral and fungal pathogens.

In addition, although many of the described examples show particular efficacy against respiratory conditions associated with cystic fibrosis, the compositions and methods described herein need not be necessarily limited to a cystic fibrosis application. For example, at least in some embodiments the compositions and methods described herein may be utilized to treat a patient with a respiratory infection even though the patient does not suffer from cystic fibrosis.

### Nonionic Metal Nanoparticles

In some embodiments, the metal nanoparticles may comprise or consist essentially of nonionic, ground state metal nanoparticles. Examples include spherical-shaped metal nanoparticles, coral-shaped metal nanoparticles, or a blend of spherical-shaped metal nanoparticles and coral-shaped metal nanoparticles. Preferred embodiments comprise spherical-shaped nanoparticles.

In some embodiments, metal nanoparticles useful for making nanoparticle compositions comprise spherical nanoparticles, preferably spherical-shaped metal nanoparticles having a solid core. The term “spherical-shaped metal nanoparticles” refers to nanoparticles that are made from one or more metals, preferably nonionic, ground state metals, having only internal bond angles and no external edges or bond angles, in contrast to hedron-like, faceted, or crystalline nanoparticles which are often formed using conventional chemical synthesis methods, even though such nanoparticles are often loosely described in the art as being “spherical” in shape.

The nonionic, spherical nanoparticles are highly resistant to ionization, highly stable, and highly resistance to agglomeration. Such nanoparticles can exhibit a high ζ-potential, which permits the spherical nanoparticles to remain dispersed within a polar solvent without a surfactant, even in the absence of a separate anti-agglomeration coating agent, which is a surprising and unexpected result.

In some embodiments, spherical-shaped metal nanoparticles can have a diameter of about 40 nm or less, about 35 nm or less, about 30 nm or less, about 25 nm or less, about 20 nm or less, about 15 nm or less, about 10 nm or less, about 7.5 nm or less, or about 5 nm or less.

In some embodiments, spherical-shaped nanoparticles can have a particle size distribution such that at least 99% of the nanoparticles have a diameter within 30% of the mean diameter of the nanoparticles, or within 20% of the mean diameter, or within 10% of the mean diameter. In some embodiments, spherical-shaped nanoparticles can have a mean particle size and at least 99% of the nanoparticles have a particle size that is within ±3 nm of the mean diameter, ±2 nm of the mean diameter, or ±1 nm of the mean diameter. The mean diameter and/or particle size distribution may be measured using techniques known in the art, such as dynamic light scattering techniques, microscopy techniques (e.g. TEM, SEM) and may be based on either a number or volume distribution.

In some embodiments, spherical-shaped nanoparticles can have a ζ-potential (measured as an absolute value) of at least 10 mV, preferably at least about 15 mV, more preferably at least about 20 mV, even more preferably at least about 25 mV, and most preferably at least about 30 mV.

Examples of laser-ablation methods and systems for manufacturing spherical-shaped nanoparticles are disclosed in U.S. Pat. No. 9,849,512 to William Niedermeyer, which is incorporated herein by this reference.

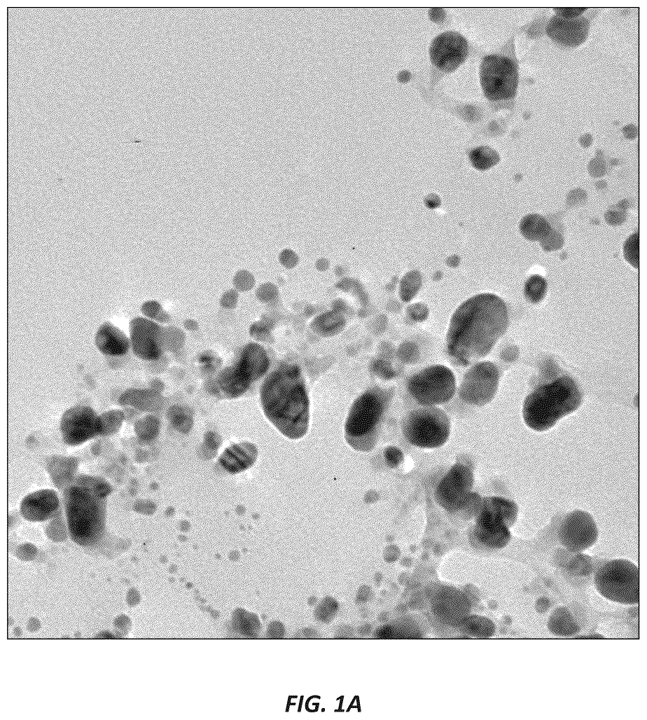

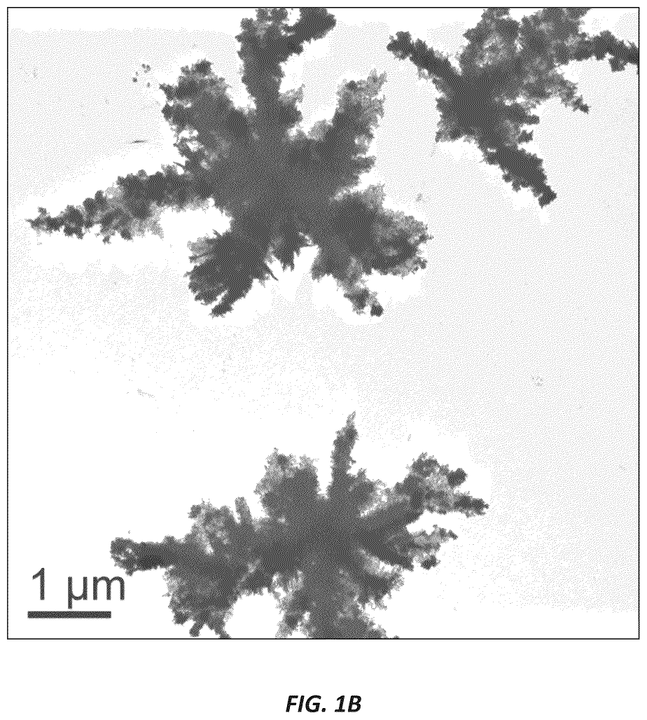

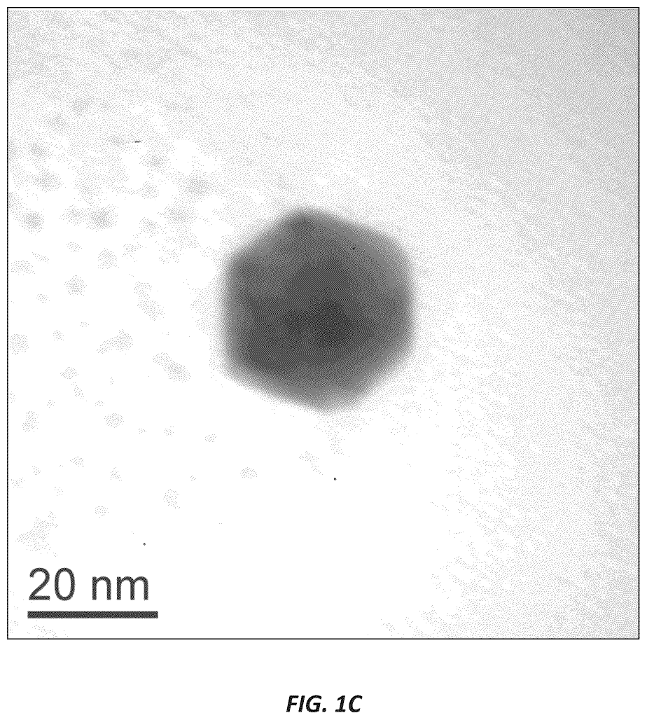

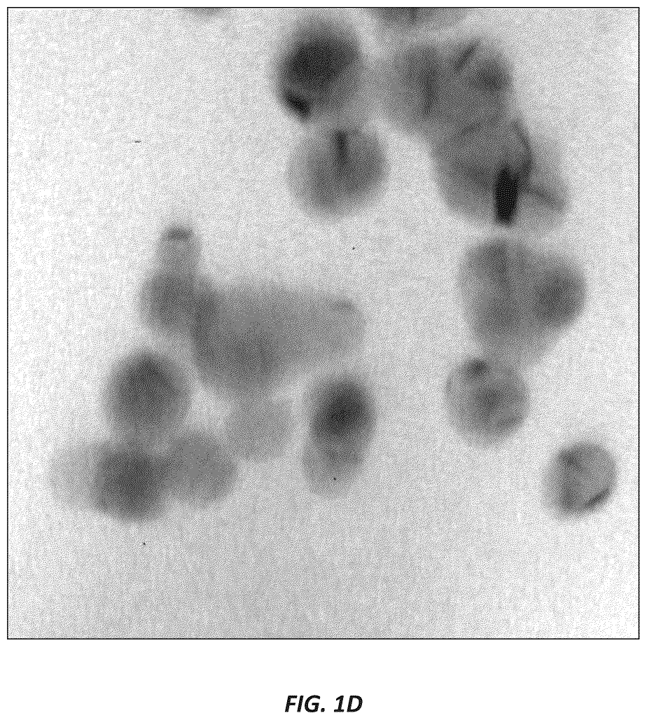

FIGS. 1A-1D show transmission electron microscope (TEM) images of nanoparticles made according to various chemical synthesis methods. As shown, the nanoparticles formed using these various chemical synthesis methods tend to exhibit a clustered, crystalline, faceted, or hedron-like shape rather than a true spherical shape with round and smooth surfaces.

For example, FIG. 1A shows silver nanoparticles formed using a common trisodium citrate method. The nanoparticles are clustered and have a relatively broad size distribution. FIG. 1B shows another set of silver nanoparticles (available from American Biotech Labs, LLC) formed using another chemical synthesis method and showing rough surface morphologies with many edges. FIG. 1C shows a gold nanoparticle having a hedron shape as opposed to a truly spherical shape. FIG. 1D shows a set of silver nanoparticles (sold under the trade name MesoSilver), which have relatively smoother surface morphologies but are understood to be shells of silver formed over a non-metallic seed material.

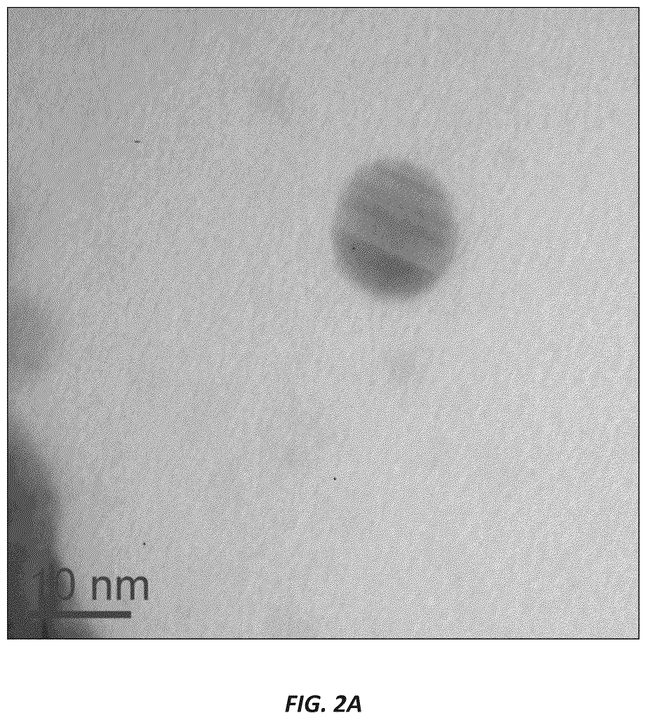

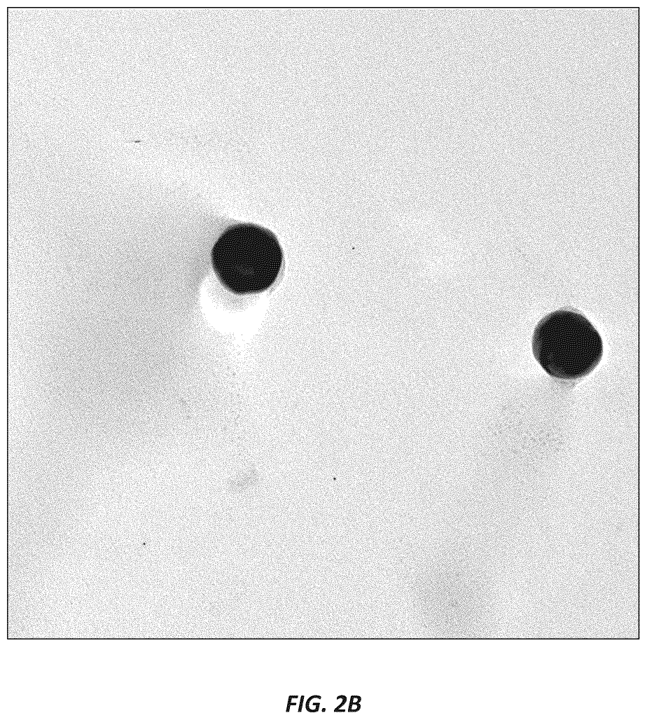

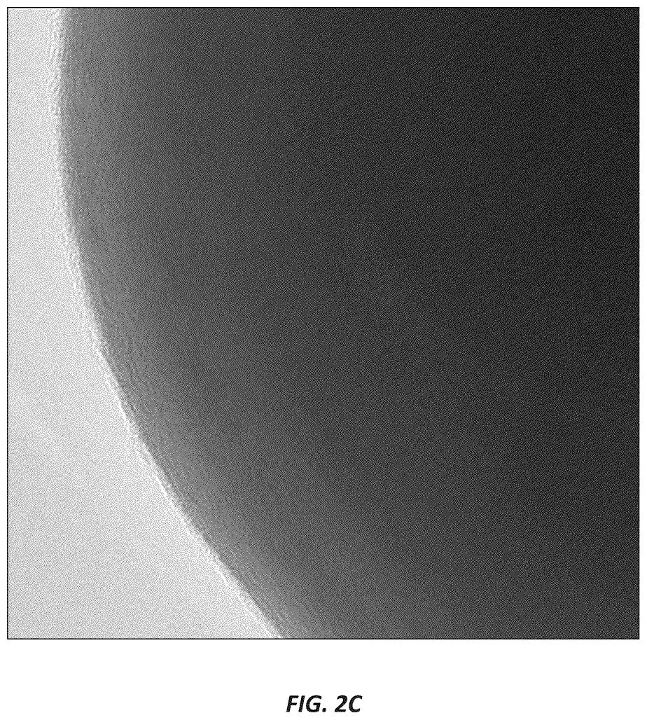

In contrast, the spherical-shaped nanoparticles described herein are solid metal, substantially unclustered, optionally exposed/uncoated, and have a smooth and round surface morphology along with a narrow size distribution. FIGS. 2A-2C show additional TEM images of spherical-shaped nanoparticles. FIG. 2A shows a gold/silver alloy nanoparticle (90% silver and 10% gold by molarity). FIG. 2B shows two spherical nanoparticles side by side to visually illustrate size similarity. FIG. 2C shows a surface of a metal nanoparticle showing the smooth and edgeless surface morphology.

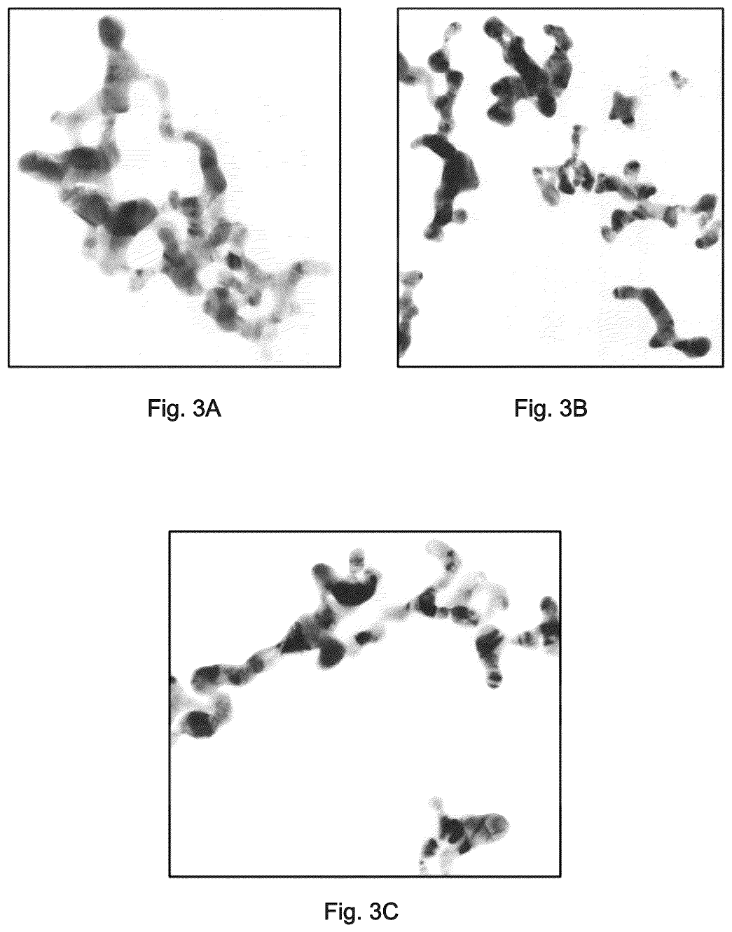

In some embodiments, nonionic metal nanoparticles useful for making nanoparticle compositions may also comprise coral-shaped nanoparticles. The term “coral-shaped metal nanoparticles” refers to nanoparticles that are made from one or more metals, preferably nonionic, ground state metals having a non-uniform cross section and a globular structure formed by multiple, non-linear strands joined together without right angles (see FIGS. 3A-3C). Similar to spherical-shaped nanoparticles, coral-shaped nanoparticles may have only internal bond angles and no external edges or bond angles. In this way, coral-shaped nanoparticles can be highly resistant to ionization, highly stable, and highly resistance to agglomeration. Such coral-shaped nanoparticles can exhibit a high-potential, which permits the coral-shaped nanoparticles to remain dispersed within a polar solvent without a surfactant, which is a surprising and unexpected result.

In some embodiments, coral-shaped nanoparticles can have lengths ranging from about 15 nm to about 100 nm, or about 25 nm to about 95 nm, or about 40 nm to about 90 nm, or about 60 nm to about 85 nm, or about 70 nm to about 80 nm. In some embodiments, coral-shaped nanoparticles can have a particle size distribution such that at least 99% of the nanoparticles have a length within 30% of the mean length, or within 20% of the mean length, or within 10% of the mean length. In some embodiments, coral-shaped nanoparticles can have a &-potential of at least 10 mV, preferably at least about 15 mV, more preferably at least about 20 mV, even more preferably at least about 25 mV, and most preferably at least about 30 mV.

Examples of laser-ablation methods and systems for manufacturing coral-shaped nanoparticles are disclosed in U.S. Pat. No. 9,919,363 to William Niedermeyer, which is incorporated herein by this reference.

The metal nanoparticles, including spherical-shaped and/or coral-shaped nanoparticles, may comprise any desired metal, mixture of metals, or metal alloy, including at least one of silver, gold, platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, or alloys thereof. Preferred embodiments comprise silver nanoparticles.

### Treatment of Respiratory Infections

The treatment compositions described herein may be used for treating a respiratory infection, and in particular for treating a respiratory infection associated with cystic fibrosis. Beneficially, the nanoparticles may be configured in size and shape to promote effective penetration of mucus in order to reach bacteria within the mucus and in order to reach underlying respiratory tissue.

In some embodiments, the nanoparticles are spherical and have a mean diameter of about 1 nm to about 40 nm, or about 2 nm to about 20 nm, or about 3 nm to about 15 nm, or about 4 nm to about 12 nm, or about 6 nm to about 10 nm, or a size range with endpoints defined by any two of the foregoing values. Nanoparticles within these size ranges, in particular nanoparticles having a mean diameter of about 8 nm, have been found to effectively penetrate mucus while still being capable of effective clearance from the patient's body (e.g., via the lymphatic system and kidneys).

The nanoparticles may be provided in an amount such that when mixed with the carrier, the nanoparticles have a concentration of about 10 ppb to about 100 ppm, or about 50 ppb to about 50 ppm, or about 200 ppb to about 20 ppm, or about 500 ppb to about 10 ppm, or about 1 ppm, or a concentration within a range defined by any two of the foregoing values.

Within these concentration ranges, the nanoparticles have been found to be effective in killing or deactivating targeted microbes. Beneficially, because the nanoparticles are effective even at relatively low concentrations, more dilute doses may be administered (and/or less nanoparticles may be dosed overall), which lowers the clearance burden on the body and reduces the risk of unwanted side-effects such as harm to the patient's own cells/tissues or systemic harm to other beneficial microbiota of the patient.

The carrier may be any pharmaceutically acceptable liquid or solid (e.g., powder) amenable to administration via inhalation. In one embodiment, the carrier comprises a saline solution. The carrier may optionally include one or more excipients suitable for use in an inhalation application. Suitable excipients include, for example, inhalable bulking powders, carbohydrates such as monosaccharides (e.g., glucose, arabinose), disaccharides (e.g., lactose, saccharose, maltose), and oligo-and polysaccharides (e.g., dextran, cyclodextrins), alcohols and polyalcohols (e.g., ethanol, sorbitol, mannitol, xylitol), salts (e.g., sodium chloride, calcium carbonate, carboxylic acid salts, fatty acid salts), amino acids (e.g., glycine), buffers (e.g., citrate, phosphate, acetate), or combinations thereof.

The treatment composition may be administered using any suitable inhalation route, including through the use of a metered-dose inhaler, a nebulizer, and/or a dry powder dispersion device. These types of devices typically include a mouthpiece or facemask enabling transfer of nebulized/atomized medicament to the patient. A nebulizer may be an ultrasonic nebulizer, a jet nebulizer, a vibrating mesh nebulizer, or a soft mist inhaler, for example.

The treatment compositions have shown versatile efficacy in treating a wide variety of bacteria, including several problematic bacterial strains that have resistance to one or more conventional antibiotics. In some embodiments, the respiratory infection may be associated with one or more of: Staphylococcus aureus (e.g., including methicillin-resistant Staphylococcus aureus), Escherichia coli, Listeria, Salmonella, Pseudomonas (e.g., including mucoid and non-mucoid Pseudomonas and/or meropenem-resistant Pseudomonas), nontuberculosis mycobacteria (e.g., including Mycobacterium abscessus complex and Mycobacterium avium complex), Acinetobacter, Strenotrophomonas (e.g., Strenotrophomonas maltophilia), Achromobacter, and Burkholderia cepacia complex (e.g., including one or more of Burkholderia cenocepacia, Burkholderia multivorans, and Burkholderia dolosa), for example.

The treatment compositions have also shown efficacy in treating various pathogenic fungi sometimes associated with respiratory infections. In some embodiments, a respiratory infection may by associated with one or more of Aspergillus (e.g., Aspergillus niger), Fusarium (e.g., Fusarium solani complex), Coccidioides, Histoplasma, Pneumocystis (e.g., Pneumocystis jirovecii), Cryptococcus (e.g., Cryptococcus neoformans, Cryptococcus gatti), Candida (e.g., Candida albicans), and Blastomyces, for example.

The treatment composition may also have efficacy in killing or deactivating viruses sometimes associated with respiratory infections, such as influenza virus, rhinovirus, respiratory syncytial virus (RSV), parainfluenza virus, adenoviruses, herpes, and rotavirus, for example.

### Antimicrobial Activity

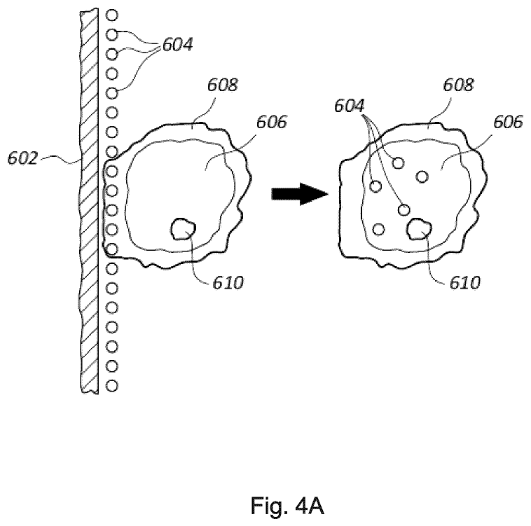

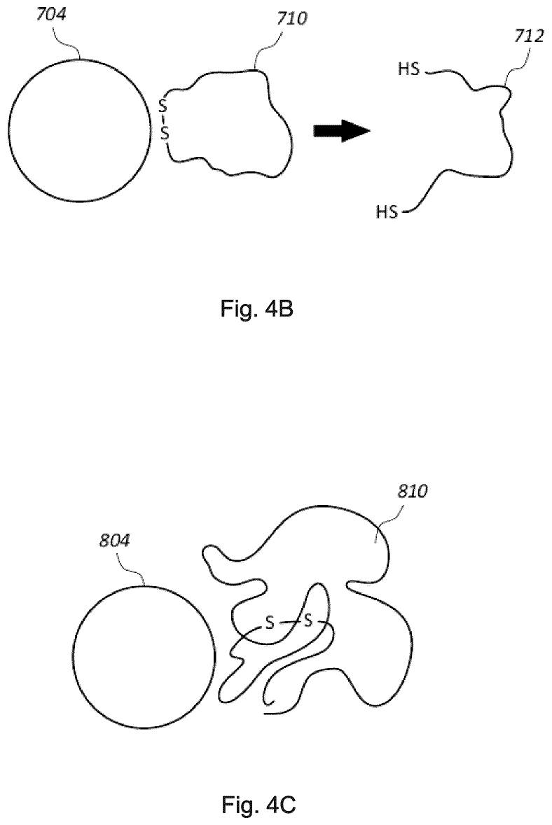

FIG. 4A schematically illustrates a bacterium 608 having absorbed spherical-shaped nanoparticles 604 from a substrate 602 (e.g., from a mucus layer), such as by active absorption or other transport mechanism. The nanoparticles 604 can freely move throughout the interior 606 of bacterium 608 and come into contact with one or more vital proteins or enzymes 610 that, if denatured, will kill or disable the bacterium. A similar mechanism may function where viral or fungal pathogens are involved. Unlike most conventional antibiotics, the nanoparticles effectively kill or deactivate the bacterium without significantly disrupting the cell wall and therefore without significant lysing of the bacteria coming into contact with the nanoparticles.

For example, one way that nanoparticles may kill or denature a microbe is by catalyzing the cleavage of disulfide (S—S) bonds within a vital protein or enzyme. FIG. 4B schematically illustrates a microbe protein or enzyme 710 with disulfide bonds being catalytically denatured by an adjacent spherical-shaped nanoparticle 704 to yield denatured protein or enzyme 712. In the case of bacteria or fungi, the cleavage of disulfide bonds and/or cleavage of other chemical bonds of vital proteins or enzymes may occur within the cell interior and thereby function to kill the microbe in this manner without causing significant lysis. Such catalytic cleavage of disulfide (S—S) bonds is facilitated by the generally simple protein structures of microbes, in which many vital disulfide bonds are on exposed and readily cleaved by catalysis.

Another potential mechanism by which metal (e.g., silver) nanoparticles can kill microbes is through the production of active oxygen species, such as peroxides, which can oxidatively cleave protein bonds, including but not limited to amide bonds.

Notwithstanding the lethal nature of nonionic metal nanoparticles relative to microbes, they can be relatively harmless to humans, mammals, and healthy mammalian cells, which contain much more complex protein structures compared to simple microbes in which most or all vital disulfide bonds are shielded by other, more stable regions of the protein. FIG. 4C schematically illustrates a mammalian protein 810 with disulfide (S—S) bonds that are shielded so as to resist being catalytically denatured by an adjacent spherical-shaped nanoparticle 804. In many cases the nonionic nanoparticles do not interact with or attach to human or mammalian cells and can be quickly and safely expelled through the urine without damaging kidneys or other cells, tissues, or organs.

The metal nanoparticles kill bacteria without significant release of silver (Ag+) or other metal ions. Because the metal nanoparticles do not release significant quantities of silver or other metal ions, they are essentially non-toxic to humans and other animals (i.e., whatever amount or concentration of ions, if any, that are released from the metal nanoparticles is/are below a threshold toxicity level at which they become toxic to humans, other mammals, birds, reptiles, fish, and amphibians).

In the particular case of silver (Ag) nanoparticles, the interaction of the silver (Ag) nanoparticle(s) within a microbe has been demonstrated to be particularly lethal without the need to rely on the production of silver ions (Ag⁺) to provide the desired antimicrobial effects, as is typically the case with conventional colloidal silver compositions. The ability of silver (Ag) nanoparticles to provide effective microbial control without any significant or actual release of toxic silver ions (Ag⁺) into the patient or the surrounding environment is a substantial advancement in the art. Whatever amount or concentration of silver ions released by silver nanoparticles, if any, is well below known or inherent toxicity levels for animals, such as mammals, birds, reptiles, fish, and amphibians.

As used herein, the modifying term “significant” means that the effect the term is modifying is clinically noticeable and relevant. Thus, the phrase “without significant release of silver ions” means that though there may technically be some small amount of detectable ion release, the amount is so small as to be clinically and functionally negligible. Similarly, the phrase “without significant cell lysis” means that although there may be some observable cell lysis, the amount is negligible and only tangentially related to the actual primary mechanism of cell death/deactivation.

### EXAMPLES

In the following examples, the nonionic, ground state, uncoated metal nanoparticles described above may be referred to as “Attostat” nanoparticles, “Niedermeyer” nanoparticles, “Attostat Ag,” or the like. Except where noted otherwise, the Attostat nanoparticles utilized were spherical, silver nanoparticles having a size of about 4 nm to about 12 nm, or more typically about 6 nm to about 10 nm.

### Example 1

Testing measured the transepithelial electrical resistance (TER) of a nanoparticle composition applied to the apical surface of cystic fibrosis patient derived primary cultures of bronchial epithelia (maintained in ALI cultures). TER is a measure of epithelial tight junction integrity which underlies the physical barrier function of airway epithelia. Changes in TER of 12 epithelia were observed over 24 hours.

Changes in measured TER of epithelia treated with spherical, nonionic, ground state silver nanoparticle formulations at 3 ppm were not significantly different from the responses to vehicle treatment at the sampled time points, as determined with ANOVA and Turkey-Kramer HSD post-test analysis with P<0.05. The impact of the silver nanoparticle formulations on the barrier function of well differentiated primary CF bronchial epithelia was therefore not distinct from the impact of vehicle treatment.

Further, there were no visually distinct differences in microscopic appearance of the epithelia at about 100× magnification in a phase contrast microscope. Ciliary activity was also similar across treatment groups. These results were surprising given the general belief that silver nanoparticles of such size would release silver ions and be toxic to such cells.

### Example 2

This test compared the effect on zebra fish of nonionic, ground state silver nanoparticles formed via laser ablation compared to other silver nanoparticles formed through conventional chemical synthesis or electrolysis methods, silver nitrate, and a control tank with plain water. The nanoparticles formed through a chemical synthesis process and the nanoparticles formed through an electrolysis process both caused the fish to exhibit signs of toxicity, including death, slowed movement and settling near the bottom of the tank. The nanoparticles formed through an electrolysis process and the silver nitrate both killed the fish within 2 hours of exposure.

In contrast, the fish in the tank treated with the nonionic, ground state silver nanoparticles of the present application and the fish in the control tank were equally healthy and active. None of the zebrafish exposed to the nonionic, ground state silver nanoparticles of the present application died during the course of the study, whereas all other treatments were associated with at least some zebrafish death.

The results of the zebrafish study were surprising in light of the general knowledge that silver nanoparticles show toxicity in such studies. For example, the authors of Mansouri et al., “Effects of Short-Term Exposure to Sublethal Concentrations of Silver Nanoparticles on Histopathology and Electron Microscope Ultrastructure of Zebrafish (Danio Rerio) Gills,” Iranian J. Toxicity, Vol. 10, No 1, January-February 2016, state the concern that “[t] he increasing use of nanomaterials and nanoproducts has increased the possibility of contamination of the environment, which may have adverse effects on different organisms” (Abstract). The authors concluded, following the study, that “[b] ased on the adverse effects of AgNPs [silver nanoparticles] on zebrafish gills, silver nanoparticle solutions can be hazardous pollutants for the environment” (page 15).

### Example 3

Neutrophil testing was done using complete blood count (CBC) analysis. Testing 0.2 and 1.0 μg/mL (i.e., ppm) of Attostat Ag showed that after up to 6 hours exposure time, no blood panel values deviated from normal ranges. At 24 hours exposure time, both control and test samples showed borderline values for MCHC (Mean Corpuscular Hemoglobin Concentration, just below minimum normal range) and MPV (Mean Platelet Volume, just above the maximum normal range).

The only deviation from normal values occurred in the 0.2 μg/mL Attostat Ag sample, which exhibited very slight elevation in EOS % (Eosinophil Percentage, just above maximum normal range). Overall, these results show no significant toxicological effects on the full spectrum of blood cells and components. This is particularly encouraging as forecasted therapeutics typically would not exceed 8-10 μg/mL, resulting in much lower local concentrations throughout the bloodstream and other portions of the body.

### Example 4

Antimicrobial efficacy tests were performed using 0.5 μg/mL Attostat Ag against five common bacterial strains associated with respiratory infections of cystic fibrosis patients:
Staphylococcus aureus
MRSA
E. coli
Listeria
Salmonella

Staph and MRSA both had >99% kill within 24 hours. E. coli, Listeria, and Salmonella both had >99% kill in approximately 12 hours.

### Example 5

A certified Tobramycin resistant strain of Pseudomonas aeruginosa was acquired from the University of Michigan and subjected to GLP Time Kill Studies. At an 0.8 μg/mL overall exposure level of Attostat Ag, the study yielded results proving high efficacy, >99%, within 1 hour of exposure.

### Example 6

Following successful results against tobramycin-resistant Pseudomonas,similar testing with B. cepacia complex (BCC) was performed. Samples of two of the most widespread strains, Burkholderia cenocepacia and Burkholderia multivorans,were obtained. Cultures of these BCC species were subjected to GLP Time Kill Studies. Attostat Ag proved highly effective against the strains with >99% kill within 1 hour of exposure for B. cenocepacia and >97% kill for B. multivorans within 1 hour of exposure (0.8 μg/mL exposure level).

### Example 7

Efficacy tests similar to those of Examples 5 and 6 were performed to compare the efficacy of Attostat Ag to tobramycin. Testing showed equal colony reduction using 4 μg/mL Attostat Ag vs 20 μg/mL tobramycin. Increasing Attostat Ag concentration to 6 μg/mL had greater colony reduction to 20 μg/mL tobramycin. Table 1 summarizes testing results from Examples 4 through 7.

TABLE 1

| | | Average | | Percent | |
|---|---|---|---|---|---|
| | | Control | Average | Reduc- | Log₁₀ |
| | Exposure | Titer | Test Titer | tion | Reduc- |
| Organism | (hours) | (CFU/ml) | (CFU/ml) | (%) | tion |
| Staphylococcus | 6 | 4.8 × 10⁶ | 1.5 × 10⁶ | 68 | 0.49 |
| aureus | 12 | | 3.7 × 10⁴ | 99.23 | 2.11 |
| | 24 | | 9.9 × 10² | 99.979 | 3.68 |
| Methicillin | 6 | 5.3 × 10⁶ | 4.4 × 10⁶ | 17 | 0.08 |
| Resistant S. | 12 | | 1.2 × 10⁴ | 78 | 0.66 |
| aureus | 24 | | 1.1 × 10⁴ | 99.8 | 2.7 |
| (MRSA) | | | | | |
| Escherichia | 6 | 9.5 × 10⁶ | 1.5 × 10⁶ | 84 | 0.79 |
| coli | 12 | | 1.3 × 10⁴ | 99.86 | 2.86 |
| | 24 | | <2.0 × 10¹ | 99.999 | >5.86 |
| Listeria | 6 | 7.0 × 10⁷ | ~1.0 × 10⁷ | ~86 | ~0.84 |
| monocytogenes | 12 | | 2.7 × 10⁶ | 96.2 | 1.42 |
| | 24 | | 9.1 × 10³ | 99.987 | 3.88 |
| Salmonella | 6 | 2.9 × 10⁷ | 1.5 × 10⁴ | 99.949 | 3.29 |
| enterica | 12 | | ~8.0 × 10¹ | ~99.999 | ~5.56 |
| | 24 | | <2.0 × 10¹ | >99.999 | >6.16 |
| Tobramycin | Control: 1 | 2.0 × 10⁶ | 1.7 × 10⁶ | 15 | 0.07 |
| Resistant P. | 1 | | 3.3 × 10¹ | 99.999 | 4.78 |
| aeruginosa | 6 | | <2.0 × 10¹ | >99.999 | >5.00 |
| | 12 | | <2.0 × 10¹ | >99.999 | >5.00 |
| | 24 | | <2.0 × 10¹ | >99.999 | >5.00 |
| Burkholderia | Control: 1 | 3.9 × 10⁶ | 2.1 × 10⁶ | 47 | 0.27 |
| cenocepacia | 1 | | 1.3 × 10³ | 99.97 | 3.46 |
| | 6 | | ~4.8 × 10² | ~99.988 | ~3.91 |
| | 12 | | ~8.7 × 10¹ | ~99.998 | ~4.65 |
| | 24 | | ~2.3 × 10¹ | ~99.999 | ~5.22 |
| Burkholderia | Control: 1 | 2.5 × 10⁶ | 1.9 × 10⁸ | 25 | 0.13 |
| multivorans | 1 | | 6.2 × 10⁶ | 97.5 | 1.61 |
| | 6 | | ~1.8 × 10³ | ~99.999 | ~5.14 |
| | 12 | | ~3.0 × 10¹ | ~99.999 | ~6.93 |
| | 24 | | ~2.0 × 10¹ | ~99.999 | ~7.10 |

### Example 8

Sputum testing was also performed using sputum donated by two individuals diagnosed with cystic fibrosis. Both patients suffer from tobramycin-resistant Pseudomonas. Initial antimicrobial efficacy testing involved culturing sputum samples in Buffered Peptone Water (BPW) overnight at various dilutions. Cultures were then used to dose well plates with anywhere from 0-10 μg/mL Attostat Ag. After 24 hours, samples treated with Attostat Ag exhibited 95-99+% bacterial kill in all cases.

### Example 9

An immunocompromised cancer patient undergoing chemotherapy and radiation therapy contracted a Fusarium fungal infection of the nasal cavity. Under the care of the supervising physician, the patient was treated with Attostat Ag via nasal inhaler. The Fusarium infection was cured following treatment.

### Example 10

Lyophilized quality control organisms were re-hydrated and grown for isolation on agar plates as indicated by the supplier in Tryptic Soy Broth or other appropriate medium and incubated. If needed, the resulting suspension was diluted in an appropriate medium so as the final concentration of the organism in the product being challenged falls between 1.0×10⁵ and 1.0×10⁶.

The product was partitioned in to 20 g aliquots in to which 100 μl of test organism was added to yield a target concentration of—5.0×10⁵ organisms per mL of product. After thoroughly mixed, each sample cup was allowed to sit for the time intervals indicated in the attached report, at which point 1.0 g aliquots were taken and diluted 1:10, with further dilutions performed as necessary. Each tube was thoroughly vortexed. From each dilution, 1mL aliquots of solution were removed and plated on to Tryptic Soy Agar plates (or other appropriate media), and then incubated under the conditions appropriate for each test organism. Following the appropriate incubation period, colony counts were taken and reported.

Log reduction values were calculated as: Log Reduction=log₁₀ (A/B), where A is the number of viable microorganisms before treatment and B is the number of viable microorganisms after treatment and time interval. Where no colonies were observed on the plates, results of less than (<) the minimum detection limit (ie. <10 cfu's/g) were reported. In these cases, the log reduction was calculated based on the minimum detection limit and reported as a greater than value. Results are summarized in Table 2.

TABLE 2

| | | | | Day 1 Log | Day 3 Log |
|---|---|---|---|---|---|
| Organism | Initial | Day 1 | Day 3 | Reduction | Reduction |
| Aspergillus niger | 1.5 × 10⁵ | 500 | 150 | 2.48 | 3.00 |
| ATCC 16404 | | | | | |
| Penicillium | 4.0 × 10⁵ | 400 | 350 | 3.00 | 3.06 |
| rubens | | | | | |
| ATCC 9179 | | | | | |

### Example 11

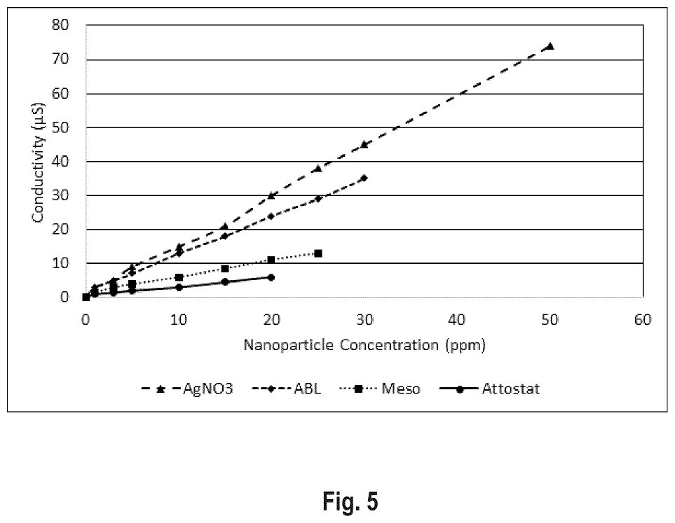

FIG. 5 illustrates the results of conductivity testing comparing various nanoparticle solutions. In Exhibit A, “Attostat” corresponds to spherical-shaped, nonionic silver nanoparticles formed by laser ablation such as described herein,

“AgNO₃” is silver nitrate, “Meso” represents a commercially available silver nanoparticle formulation with nanoparticles formed through a chemical reduction process, and “ABL” represents a commercially available silver nanoparticle formulation understood to be formed through an electrolysis process.

The results illustrate that the Attostat nanoparticle formulation had significantly less ion release than any of the other tested nanoparticle formulations. It should be noted that the measured conductivity for Attostat nanoparticle formulations, even at the highest measured concentration of 16 ppm, remained low enough to be on par with typical conductivity measurements for high quality deionized water.

### Example 12

An antibacterial efficacy test was carried out comparing a “Niedermeyer” nanoparticle formulation (8 nm size) against silver nitrate and against the National Institute of Standards and Technology (NIST) Standard Nanocomposix 10 nm silver nanoparticles. The NIST nanoparticles are formed by a chemical reduction process that utilizes citrate as reducing and capping agent. The NIST nanoparticles have a conductivity similar to the “Meso” nanoparticles of Example 11, with detectable but low levels of silver ions.

Relative Light Unit (RLU) counts were recorded at 12 hours and 24 hours post treatment. RLU measurements were carried out using a Hygiena SystemSURE Plus V.2 SN067503 RLU meter with Hygenia AquaSnap TOTAL ATP Water Test Cat #U143 Lot #153019. Culturing media was Hardy Diagnostics Buffered Peptone Water Lot #118272. Samples were prepared with the nanoparticle treatments and then diluted with the media to provide the tested concentrations. The test organism (Microbiologics, E. coli, KwikStik, ATCC #51813, Ref #0791 K, Lot #791-1-6) was incubated in fresh Buffered Peptone Water growth media for 24 hours prior to exposure to the nanoparticle treatments. Tables 3 and 4 illustrate results of RLU counts 12 and 24 hours post nanoparticle treatment, respectively.

TABLE 3
RLU Counts at 12 Hours Post Exposure to Nanoparticle Treatment

| | Attostat 8 nm | NIST Standard | AgNO3 Silver |
|---|---|---|---|
| Concentration | Particles | Particles 10 nm | Nitrate |
| Control 0 ppm (mg/L) | 6256 | 7037 | 6731 |
| 0.25 ppm (mg/L) | 65 | 6908 | 80 |
| 0.5 ppm (mg/L) | 72 | 5416 | 75 |
| 1.0 ppm (mg/L) | 30 | 7189 | 84 |

TABLE 4
RLU Counts at 24 Hours Post Exposure to Nanoparticle Treatment

| | Attostat 8 nm | NIST Standard | AgNO3 Silver |
|---|---|---|---|
| Concentration | Particles | Particles 10 nm | Nitrate |
| Control 0 ppm (mg/L) | 7595 | 5421 | 7342 |
| 0.25 ppm (mg/L) | 25 | 5691 | 25 |
| 0.5 ppm (mg/L) | 8 | 3950 | 46 |
| 1.0 ppm (mg/L) | 30 | 3834 | 30 |

Tables 5 and 6 represent the data in terms of comparing each treatment to its respective control at 12 and 24 hours post treatment, respectively.

TABLE 5
RLU as percentage of control at 12 Hours Post Treatment

| | Attostat 8 nm | NIST Standard | AgNO3 Silver |
|---|---|---|---|
| Concentration | Particles | Particles 10 nm | Nitrate |
| Control 0 ppm (mg/L) | 100% | 100% | 100% |
| 0.25 ppm (mg/L) | 1.0% | 98.2% | 1.2% |
| 0.5 ppm (mg/L) | 1.1% | 77.0% | 1.1% |
| 1.0 ppm (mg/L) | 0.6% | 102.2% | 1.3% |

TABLE 6
RLU as percentage of control at 24 Hours Post Treatment

| | Attostat 8 nm | NIST Standard | AgNO3 Silver |
|---|---|---|---|
| Concentration | Particles | Particles 10 nm | Nitrate |
| Control 0 ppm (mg/L) | 100% | 100% | 100% |
| 0.25 ppm (mg/L) | 0.33% | 105% | 0.34% |
| 0.5 ppm (mg/L) | 0.11% | 72.9% | 0.62% |
| 1.0 ppm (mg/L) | 0.39% | 70.7% | 0.41% |

As shown, at all concentrations tested, the Attostat nanoparticles reduced the number of RLU counts to less than 1.5% from the control baseline at both the 12 hour and 24 hour measurement periods. Anything below 1.5% is below level of accurate detection and is considered a complete kill.

The Attostat nanoparticles effectively reduced RLU counts to below the 1.5% threshold at all tested concentrations. The NIST nanoparticles appeared to show a trend toward greater efficacy at higher concentrations, which would correspond to a normal diffusion model, but even at the highest tested concentration still only reached an RLU count of 70.7% of the initial control baseline at the 24 hour measurement.

The low antimicrobial efficacy of the NIST nanoparticles at the concentrations tested as compared to the silver nitrate could potentially be explained by the lower conductivity, and thus lower ion concentration, of the NIST nanoparticles as compared to the silver nitrate. However, the significant efficacy of the Attostat nanoparticles was surprising given the fact that the Attostat nanoparticles have significantly low to non-detectable levels of ions, even lower than the NIST particles.

The Attostat nanoparticles continued to provide antimicrobial activity through the 24 hour testing period with no signs of reduced efficacy.

1. A method for treating a respiratory infection, comprising:
administering a treatment composition to a patient in need thereof via inhalation into the patient's lungs, with the proviso that the respiratory infection is not caused by methicillin-resistant Staphylococcus aureus or Pneumocystis, wherein the treatment composition comprises
nonionic, ground state, spherical silver nanoparticles having a mean diameter in a range of about 1 nm to about 40 nm and a particle size distribution such that at least 99% of the silver nanoparticles have a diameter within 30% of the mean diameter, wherein the nanoparticles are non-crystalline with no external edges or bond angles, and
a carrier formulated for administration via inhalation; and
the treatment composition treating the respiratory infection by the silver nanoparticles killing or deactivating microbes associated with the respiratory infection without the release of silver ions.

2. The method of claim 1, wherein the treatment composition penetrates mucus of the patient to kill or deactivate microbes within the mucus and infected respiratory tissue.

3. The method of claim 1, wherein the respiratory infection is or includes a bacterial infection.

4. The method of claim 3, wherein the bacterial infection comprises biofilm, the treatment composition killing bacteria in the biofilm.

5. The method of claim 3, wherein the bacterial infection is associated with one or more bacteria selected from Escherichia coli, Listeria, Salmonella, Pseudomonas, nontuberculosis mycobacteria, Mycobacterium abscessus complex, Mycobacterium avium complex, Acinetobacter, Strenotrophomonas maltophilia, Achromobacter, tobramycin-resistant Pseudomonas, multidrug-resistant Pseudomonas aeruginosa, Burkholderia cepacia complex, Burkholderia cenocepacia, Burkholderia multivorans, and Burkholderia dolosa.

6. The method of claim 1, wherein the respiratory infection is or includes a fungal infection.

7. The method of claim 6, wherein the fungal infection is associated with one or more fungi selected from Aspergillus, Aspergillus niger, Fusarium, Fusarium solani complex, Coccidioides, Histoplasma, Pneumocystis, Pneumocystis jirovecii, Cryptococcus, Cryptococcus neoformans, Cryptococcus gatti, Candida, Candida albicans, and Blastomyces.

8. The method of claim 1, wherein the respiratory infection is or includes a viral infection.

9. The method of claim 8, wherein the viral infection is associated with one or more viruses selected from influenza virus, rhinovirus, respiratory syncytial virus (RSV), parainfluenza virus, adenoviruses, herpes, and rotavirus.

10. The method of claim 1, wherein the treatment composition is administered to the patient via inhalation using a nebulizer or a metered-dose inhaler.

11. The method of claim 1, wherein the nanoparticles kill or deactivate microbes of the infection without damaging lung epithelia.

12. The method of claim 1, wherein the silver nanoparticles have a mean diameter in a range of about 1 nm to about 20 nm.

13. The method of claim 1, wherein the carrier comprises one or more of saline, a carbohydrate, an amino acid, a salt, a buffer, an alcohol, a polyalcohol, or mixture thereof.

14. The method of claim 1, wherein the nanoparticles are administered at a concentration of about 10 ppb to about 100 ppm.

15. A method for treating a bacterial respiratory infection, comprising:
administering a treatment composition to a patient in need thereof via inhalation into the patient's lungs, with the proviso that the bacterial respiratory infection is not caused by methicillin-resistant Staphylococcus aureus or Pneumocystis, wherein the treatment composition comprises:
nonionic, ground state, spherical silver nanoparticles having a mean diameter in a range of about 1 nm to about 40 nm and a particle size distribution such that at least 99% of the silver nanoparticles have a diameter within 30% of the mean diameter, wherein the nanoparticles are non-crystalline with no external edges or bond angles, and
a carrier formulated for administration via inhalation; and
the treatment composition treating the bacterial respiratory infection by the silver nanoparticles killing or deactivating bacteria associated with the bacterial respiratory infection in mucus, biofilm, and respiratory tissue without the release of silver ions.

16. The method of claim 15, wherein the bacterial infection is associated with one or more bacteria selected from Escherichia coli, Listeria, Salmonella, Pseudomonas, nontuberculosis mycobacteria, Mycobacterium abscessus complex, Mycobacterium avium complex, Acinetobacter, Strenotrophomonas maltophilia, Achromobacter, tobramycin-resistant Pseudomonas, multidrug-resistant Pseudomonas aeruginosa, Burkholderia cepacia, Burkholderia cenocepacia, Burkholderia multivorans, and Burkholderia dolosa.

17. The method of claim 15, wherein the patient's lungs also include a fungal or viral infection, the silver nanoparticles killing or deactivating fungi and/or viruses associated with the fungal or viral infection.

18. A method for treating a respiratory infection, comprising:
administering a treatment composition to a patient in need thereof via inhalation into the patient's lungs, with the proviso that the respiratory infection is not caused by methicillin-resistant Staphylococcus aureus or Pneumocystis, wherein the respiratory infection includes at least one of a fungal infection or a viral infection, wherein the treatment composition comprises:
nonionic, ground state, spherical silver nanoparticles having a mean diameter in a range of about 1 nm to about 40 nm and a particle size distribution such that at least 99% of the silver nanoparticles have a diameter within 30% of the mean diameter, wherein the nanoparticles are non-crystalline with no external edges or bond angles, and
a carrier formulated for administration via inhalation; and
the treatment composition treating the respiratory infection by the silver nanoparticles killing or deactivating fungi and/or viruses associated with the respiratory infection in respiratory tissue without the release of silver ions.

19. The method of claim 18, wherein the respiratory infection is or includes a fungal infection associated with one or more fungi selected from Aspergillus, Aspergillus niger, Fusarium, Fusarium solani complex, Coccidioides, Histoplasma, Pneumocystis, Pneumocystis jirovecii, Cryptococcus, Cryptococcus neoformans, Cryptococcus gatti, Candida, Candida albicans, and Blastomyces.

20. The method of claim 18, wherein the respiratory infection is or includes a viral infection associated with one or more viruses selected from influenza virus, rhinovirus, respiratory syncytial virus (RSV), parainfluenza virus, adenoviruses, herpes, and rotavirus.
